SEQUENCE LISTING (10 sequences)

SEQ ID NO: 1 - DNA, 21 bp, synthetic construct
  source 1..21: mol_type = other DNA
aggcttccagtaccattaggt

SEQ ID NO: 2 - DNA, 22 bp, synthetic construct
  source 1..22: mol_type = other DNA
ctgagtgaggcaaagctgattt

SEQ ID NO: 3 - DNA, 23 bp, synthetic construct
  source 1..23: mol_type = other DNA
tatggagtgacatagagtgtgct

SEQ ID NO: 4 - DNA, 22 bp, synthetic construct
  source 1..22: mol_type = other DNA
ccacttcaatccacccagaaag

SEQ ID NO: 5 - DNA, 19 bp, synthetic construct
  source 1..19: mol_type = other DNA
cagcacggtgaagccattc

SEQ ID NO: 6 - DNA, 17 bp, synthetic construct
  source 1..17: mol_type = other DNA
gcgtgcatccgcttgtg

SEQ ID NO: 7 - DNA, 19 bp, synthetic construct
  source 1..19: mol_type = other DNA
ccaaagaaccaagaggccc

SEQ ID NO: 8 - DNA, 21 bp, synthetic construct
  source 1..21: mol_type = other DNA
gctgctgttttctgccacact

SEQ ID NO: 9 - DNA, 20 bp, synthetic construct
  source 1..20: mol_type = other DNA
gaagtccctcaccctcccaa

SEQ ID NO: 10 - DNA, 16 bp, synthetic construct
  source 1..16: mol_type = other DNA
ggcatggacgcgacca

(19) United States
(12) Patent Application Publication
(10) Pub. No.: US 2024/0299482 A1
(43) Pub. Date: Sep. 12, 2024

(54) PROCESS AND COMPOSITIONS FOR ENHANCING ENERGY EXPENDITURE

(71) Applicants: Muhammed Majeed, Bangalore, India (IN); Kalyanam Nagabhushanam, East Windsor, NJ (US); Lakshmi Mundkur, Bangalore (IN); Mohammad Mansoor Ansari, Bangalore (IN)

(72) Inventors: Muhammed Majeed, Bangalore, India (IN); Kalyanam Nagabhushanam, East Windsor, NJ (US); Lakshmi Mundkur, Bangalore (IN); Mohammad Mansoor Ansari, Bangalore (IN)

(21) Appl. No.: 18/430,296

(22) Filed: Feb. 1, 2024

Related U.S. Application Data
(63) Continuation-in-part of application No. 17/151,124, filed on Jan. 16, 2021, now Pat. No. 11,925,671.

(51) Int. Cl. A61K 36/8905; A61K 9/00; A61K 9/20; A61K 9/48; A61K 31/05; A61K 31/343; A61K 36/185

(52) U.S. Cl. CPC A61K 36/8905; A61K 9/0053; A61K 9/2009; A61K 9/2013; A61K 9/2027; A61K 9/2054; A61K 9/2059; A61K 9/4866; A61K 31/05; A61K 31/343; A61K 36/185; A61K 2236/15; A61K 2236/33; A61K 2236/37; A61K 2236/39; A61K 2236/51; A61K 2236/55

(57) ABSTRACT

The invention discloses a composition comprising Cyperus rotundus extract and Passiflora edulis extract comprising piceatannol, scirpusin B, scirpusin A, cyperusphenol A and cyperusphenol B, standardized to contain 6-10% w/w total stilbenes and a process of preparing the composition thereof. The invention also discloses the use of the above mentioned composition in enhancing energy expenditure in mammalian adipose cellular systems by the conversion of white adipose tissues to brown adipose tissues, inhibition of adipogenesis and for preventing weight gain in mammals.

### CROSS-REFERENCE TO RELATED PATENT APPLICATIONS

This is a continuation-in-part application of non-provisional U.S. patent application Ser. No. 17/151,124 filed on 16 Jan. 2021, claiming priority from U.S. Provisional application No. 62/962,339, filed on 17 Jan. 2020, the details of which are being incorporated herein in its entirety.

### INCORPORATION BY REFERENCE

“The sequence listing in the CRF is incorporated herein by reference. The details of the CRF is below:
i) Name of the XML file: CIRPUSINS
ii) Date of Creation: 19 Jan. 2024
iii) Size: 9,564 bytes

### FIELD OF INVENTION

The present invention relates to composition for enhancing energy expenditure. More specifically, the invention pertains to compositions comprising Cyperus rotundus extract and Passiflora edulis extract for increasing energy expenditure in mammalian cells by conversion of WAT to BAT.

### BACKGROUND OF THE INVENTION

Obesity is considered to be the leading health risk for the development of various disorders like hypertension, type 2 diabetes, heart disease, stroke, osteoarthritis, and mental illness. Globally, more than 1 in 10 individuals are obese and about 36% of American adults are obese (https://www.medicalnewstoday.com/articles/319902.php, accessed on 10 May 2018). Obesity results due to imbalance between the energy content of food eaten and energy expended by the body to maintain life and to perform physical work. Such an energy balance framework is a potentially powerful tool for investigating the regulation of body weight.

The conversion of white adipose tissue (WAT) to brown adipose tissue (BAT), or termed as “browning” of WAT, is now garnering more attention to enhance energy expenditure. While, WAT serves as a store house of energy, BAT lipids serve primarily as fuel for oxidative phosphorylation, primarily depending on UCPI activity and increases energy expenditure by the production of ATP. BAT is also involved in heat production and helps in maintaining an adequate core body temperature. BAT also has deeper roles in the mammalian body system like Norepinephrine-induced thermogenesis, Metaboloregulatory Thermogenesis, as a secretory organ, in effective glucose and lipid clearance, Hibernation and Arousal etc. (Cannon et al., Physiol Rev 84: 277-359, 2004). Thus BAT, plays an important role in maintaining energy homeostasis.

In various instances, wherein energy expenditure is essential like in obesity, metabolic disorders, diabetes, cold thermogenesis, sports endurance etc, change from WAT to BAT is crucial for the maintenance of an effective energy homeostasis. Plant based dietary compounds have been reported to promote BAT activation and WAT browning. The role of brown adipose tissue (BAT) is well described in the following prior arts:
1. Hadi et al., Front. Physiol., 11 Jan. 2019, doi https://doi.org/10.3389/fphys.2018.01954
2. Elattar. S and Satyanarayana, “Can Brown Fat Win the Battle against White Fat?”, J Cell Physiol. 2015 October; 230(10):2311-7
3. Zafrir B, “Brown adipose tissue: research milestones of a potential player in human energy balance and obesity”, Horm Metab Res. 2013 October; 45(11):774-85)
4. Giralt M, Villarrova F “White, brown, beige/brite: different adipose cells for different functions?” Endocrinology. 2013 September; 154(9):2992-3000

Drugs and/or natural molecules that facilitate the conversion of white to brown adipocytes are effective in the treatment/management of obesity related conditions. US patent publication no. 20150238438 discloses a composition comprising extract of Cyperus rotundus rhizomes standardized to contain 5% of total stilbenes, consisting essentially of a combination of Piccatannol, Scirpusin B and Scirpusin A for the management of obesity. US patent publication no. 20190328818 discloses a composition comprising Cyperus rotundus extract, Garcinia extract and Coleus forskohlii extract for the management of obesity. However, the prior art documents fail to address the issue of maintaining energy homeostasis which is important in managing many disease conditions including obesity. Further, we need a better understanding of the components involved in energy expenditure and their interactions over various time scales to explain the natural history of conditions such as obesity and to estimate the magnitude and potential success of therapeutic interventions. (Kevin D. Hall, Steven B. Heymsfield, Joseph W. Kemnitz, Samuel Klein, Dale A. Schoeller, and John R. Speakman, Energy balance and its components: implications for body weight regulation, Am J Clin Nutr. 2012 April; 95(4): 989-994). The present invention solves the above problem by disclosing a composition comprising Cyperus rotundus extract and Passiflora edulis extract standardised to contain Piceatannol, Scirpusin B and Scirpusin A, Cyperusphenol A and Cyperusphenol B for enhancing energy expenditure.

The principle object of the invention is to disclose a composition comprising Cyperus rotundus extract and Passiflora edulis extract comprising Piccatannol, Scirpusin B and Scirpusin A, and Cyperusphenols, standardized to contain 6-10% w/w total stilbenes and the process of formulating the composition.

It is another object of the invention to disclose a method of enhancing energy expenditure in mammalian adipose cellular systems using a composition comprising Cyperus rotundus extract and Passiflora edulis extract comprising Piceatannol, Scirpusin B, Scirpusin A and Cyperusphenols, standardized to contain 6-10% w/w total stilbenes.

It is yet another object of the invention to disclose a method of therapeutic management of obesity in mammals using a composition comprising Cyperus rotundus extract and Passiflora edulis extract comprising Piccatannol, Scirpusin B, Scirpusin A and Cyperusphenols, standardized to contain 6-10% w/w total stilbenes.

The present invention fulfils the above mentioned objectives and provides further related advantages.

### SUMMARY OF THE INVENTION

In a most preferred embodiment, the invention discloses a composition comprising Cyperus rotundus extract and Passiflora edulis extract comprising Piccatannol, Scirpusin B, Scirpusin A, Cyperusphenol A and Cyperusphenol B, standardized to contain 6-10% w/w total stilbenes.

In a preferred embodiment, the invention discloses a method of enhancing energy expenditure in mammalian adipose cellular systems, said method comprising: step of bringing into contact mammalian pre-adipocytes or adipocytes into contact with a composition comprising Cyperus rotundus extract and Passiflora edulis extract comprising Piccatannol, Scirpusin B, Scirpusin A Cyperusphenol A and Cyperusphenol B, standardized to contain 6-10% w/w total stilbenes to achieve effects of (a) increased expression of genes and proteins that function individually or in combination to specifically recruit brown adipocytes or beige or brite adipocytes, (b) induce brown or beige or brite like phenotype in white adipocyte depots, (c) induce enhanced expression of mitochondrial uncoupling protein-1 to result in increased mitochondrial thermogenesis in differentiated brown adipocytes and beige or brite adipocytes.

In another preferred embodiment, the invention discloses a method of inhibition of adipogenesis in mammalian cellular systems, said method comprising step of bringing into contact mammalian adipocytes into contact with a composition comprising Cyperus rotundus extract and Passiflora edulis extract comprising Piccatannol, Scirpusin B, Scirpusin A Cyperusphenol A and Cyperusphenol B, standardized to contain 6-10% w/w total stilbenes to bring about inhibition in adipogenesis.

In another preferred embodiment, the invention discloses a method for therapeutic management of obesity in mammals, said the method comprising steps of administering an effective concentration of a composition comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes to said mammals to bring about prevention in weight gain.

Other features and advantages of the present invention will become apparent from the following more detailed description, taken in conjunction with the accompanying images, which illustrate, by way of example, the principle of the invention.

### DESCRIPTION OF DRAWINGS

The patent or application file contains at least one drawing executed in color. Copies of this patent or patent application publication with color drawing(s) will be provided by the office upon request and payment of the necessary fec.

FIG. 1A is a graphical representation showing the increase in expression of genes related to brown fat conversion in mouse 3T3-L1 pre-adipocytes administered with composition comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes at 3.125 μg/ml.

FIG. 1B is a graphical representation showing the increase in expression of genes related to brown fat conversion in mouse 3T3-L1 pre-adipocytes administered with composition comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes at 6.25 μg/ml.

FIG. 2A is the oil-O-red staining of adipocytes indicating a dose dependent reduction in lipid accumulation in adipocytes by comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes.

FIG. 2B is the graphical representation of the percentage inhibition of adipogenesis by composition comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes.

FIG. 3 is a graphical representation showing the weight gain in animals administered with different concentrations of composition comprising Cyperus rotundus extract and Passiflora edulis extract comprising Piccatannol, Scirpusin B, Scirpusin A Cyperusphenol A and Cyperusphenol B, standardized to contain 6-10% w/w total stilbenesover a period of 120 days.

FIG. 4 is a graphical representation showing the change in weight of animals administered with different concentrations of composition comprising Cyperus rotundus extract and Passiflora edulis extract comprising Piccatannol, Scirpusin B, Scirpusin A Cyperusphenol A and Cyperusphenol B, standardized to contain 6-10% w/w total stilbenesover a period of 120 days.

### DESCRIPTION OF PREFERRED EMBODIMENTS

In a most preferred embodiment, the invention discloses a composition comprising Cyperus rotundus extract and Passiflora edulis extract comprising Piccatannol, Scirpusin B, Scirpusin A, Cyperusphenol A and Cyperusphenol B, standardized to contain 6-10% w/w total stilbenes. In another related embodiment, the Cyperus rotundus extract is standardized to contain 0.1-5% total stilbenes and Passiflora edulis extract is standardized to contain 15-25% total stilbenes. In a related embodiment, the Cyperus rotundus extract and Passiflora edulis extract are in the ratios ranging from 1:5 to 5:1. In a related embodiment, Cyperusphenol A include both the meso and dl isomers since these co-occur in Cyperus rotundus as described by T Ito, H Endo, H Shinohara, M Oyama, Y Akao and M Iinuma, Fitoterapia 83, 1420-1429 (2012)

In another preferred embodiment, the composition comprising Cyperus rotundus extract and Passiflora edulis extract is obtained by the process comprising steps of
a) Powdering tubers of Cyperus rotundus into a fine mesh
b) Refluxing with 4 volumes of methanol and heating at 65-70° C. for 3-4 hours and filtering the solution to obtain a clear extract;
c) Repeating the process of step b) twice and combining the three extracts;
d) Concentrating the extract of step c) to obtain as extract containing 0.1-5% total stilbenes.
e) Powdering seeds of Passiflora edulis into a fine mesh
f) Refluxing with 4 volumes of hexane and heating at 65-70° C. for 3-4 hours and filtering the solution;
g) Repeating the process of step f) twice and combining the three hexane extracts;
h) Concentrating the combined extract of step g) by distilling the solvent to obtain a clear Marc;
i) Adding 4 volumes of ethanol to the Marc of step h) and heating at 70-75° C. and filtering;
j) Repeating the process of step i) twice and combining the three ethanol extracts to obtain a clear ethanol extract of the Passiflora edulis seeds;
k) Dissolving the ethanol extract of step j) in 6 volumes of water;
l) Charging with 6 volumes of ethyl acetate and stirring for 5 minutes and allowing to settle for 1 hour;
m) Collecting the ethyl acetate layer and washing with water;
n) Repeating the process of steps 1) and m) two more times and combining all ethyl acetate layers;
o) Concentrating the ethyl acetate layer by heating at 65-75° C. to obtain Passiflora edulis seed extract containing 15-25% total stilbenes.
p) Mixing the Cyperus rotundus extract and Passiflora edulis seed extract in the ratios of 1:5, 2:4, 3:3, 4:2, 2:1 and 5:1 respectively to obtain a composition comprising Cyperus rotundus extract and Passiflora edulis extract comprising Piccatannol, Scirpusin B, Scirpusin A Cyperusphenol A and Cyperusphenol B, standardized to contain 6-10% w/w total stilbenes.

In another related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In a preferred embodiment, the invention discloses a method of enhancing energy expenditure in mammalian adipose cellular systems, said method comprising: step of bringing into contact mammalian pre-adipocytes or adipocytes into contact with a composition comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes to achieve effects of (a) increased expression of genes and proteins that function individually or in combination to specifically recruit brown adipocytes or beige or brite adipocytes, (b) induce brown or beige or brite like phenotype in white adipocyte depots, (c) induce enhanced expression of mitochondrial uncoupling protein-1 to result in increased mitochondrial thermogenesis in differentiated brown adipocytes and beige or brite adipocytes. In a related embodiment, the genes and proteins are at least one factor selected from the group consisting of comprising PRDM16, PGC-1α and BMP7. In a related embodiment, enhancing energy expenditure is essential in conditions selected from the group consisting of, but not limited to, obesity, metabolic disorders, diabetes, cold thermogenesis and sports endurance

In another preferred embodiment, the invention discloses a composition comprising Cyperus rotundus extract and Passiflora edulis extract comprising Piccatannol, Scirpusin B, Scirpusin A CyperusphenolCyperusphenol A and Cyperusphenol B, standardized to contain 6-10% w/w total stilbenes for use in enhancing energy expenditure in mammalian preadipocytes or adipocytes by administering in effective amounts targeted towards mammalian pre-adipocytes to bring about the effects of (a) increased expression of genes and proteins that function individually or in combination to specifically recruit brown adipocytes or beige or brite adipocytes, (b) induce brown or beige or brite like phenotype in white adipocyte depots, (c) induce enhanced expression of mitochondrial uncoupling protein-1 to result in increased mitochondrial thermogenesis in differentiated brown adipocytes and beige or brite adipocytes. In a related embodiment, the genes and proteins are at least one factor selected from the group consisting of comprising PRDM16, PGC-1α and BMP7. In a related embodiment, enhancing energy expenditure is essential in conditions selected from the group consisting of, but not limited to, obesity, metabolic disorders, diabetes, cold thermogenesis and sports endurance.

In a preferred embodiment, the invention discloses a method of inhibition of adipogenesis in mammalian cellular systems, said method comprising step of bringing into contact mammalian adipocytes into contact with a composition comprising Cyperus rotundus extract and Passiflora edulis extract comprising Piccatannol, Scirpusin B, Scirpusin A Cyperusphenol A and Cyperusphenol B, standardized to contain 6-10% w/w total stilbenes to bring about inhibition in adipogenesis.

In yet another preferred embodiment, the invention discloses a composition comprising Cyperus rotundus extract and Passiflora edulis extract comprising Piccatannol, Scirpusin B, Scirpusin A Cyperusphenol A and Cyperusphenol B, standardized to contain 6-10% w/w total stilbenes use in inhibiting adipogenesis in mammalian adipocytes.

In another preferred embodiment, the invention discloses a method for therapeutic management of obesity in mammals, said the method comprising steps of administering an effective concentration of a composition comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes to said mammals to bring about prevention in weight gain.

In another preferred embodiment, the invention discloses a composition comprising Cyperus rotundus extract and Passiflora edulis extract comprising Piccatannol, Scirpusin B, Scirpusin A Cyperusphenol A and Cyperusphenol B, standardized to contain 6-10% w/w total stilbenes for preventing weight gain in mammals. In another related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

The aforesaid most preferred embodiments incorporating the technical features and technical effects of instant invention, are explained through illustrative examples herein under.

### Example 1: Isolation and Formulation of Composition

The composition comprising Cyperus rotundus extract and Passiflora edulis extract is obtained by the process comprising steps of
a) Powdering tubers of Cyperus rotundus into a fine mesh
b) Refluxing with 4 volumes of methanol and heating at 65-70° C. for 3-4 hours and filtering the solution to obtain a clear extract;
c) Repeating the process of step b) twice and combining the three extracts;
d) Concentrating the extract of step c) to obtain as extract containing 0.1-5% total stilbenes.
e) Powdering seeds of Passiflora edulis into a fine mesh
f) Refluxing with 4 volumes of hexane and heating at 65-70° C. for 3-4 hours and filtering the solution;
g) Repeating the process of step f) twice and combining the three hexane extracts;
h) Concentrating the combined extract of step g) by distilling the solvent to obtain a clear Marc;
i) Adding 4 volumes of ethanol to the Marc of step h) and heating at 70-75° C. and filtering;
j) Repeating the process of step i) twice and combining the three ethanol extracts to obtain a clear ethanol extract of the Passiflora edulis seeds;
k) Dissolving the ethanol extract of step j) in 6 volumes of water;
l) Charging with 6 volumes of ethyl acetate and stirring for 5 minutes and allowing to settle for 1 hour;
m) Collecting the ethyl acetate layer and washing with water;
n) Repeating the process of steps 1) and m) two more times and combining all ethyl acetate layers;
o) Concentrating the ethyl acetate layer by heating at 65-75° C. to obtain Passiflora edulis seed extract containing 15-25% total stilbenes.
p) Mixing the Cyperus rotundus extract and Passiflora edulis seed extract in the ratios of 1:5, 2:4, 3:3, 4:2, 2:1 and 5:1 respectively to obtain a composition comprising Cyperus rotundus extract and Passiflora edulis extract comprising Piccatannol, Scirpusin B, Scirpusin A Cyperusphenol A and Cyperusphenol B, standardized to contain 6-10% w/w total stilbenes.

As an illustrative example, the Cyperus rotundus extract and Passiflora edulis seed extract are mixed in the ratios of 2:1 respectively to obtain a composition comprising Cyperus rotundus extract and Passiflora edulis extract comprising Piccatannol, Scirpusin B, Scirpusin A Cyperusphenol A and Cyperusphenol B, standardized to contain 7% w/w total stilbenes. The Cyperus rotundus extract and Passiflora edulis seed extract are also mixed in the above mentioned ratios along with pharmaceutically/nutraceutically acceptable excipients, adjuvants, diluents or carriers in a way that the final formulation is standardized to contain 6-10% w/w total stilbenes.

### Example 2: Adipogenesis Inhibition and Brown Fat Specific Gene Expression by Cyperus rotundus Extract and Passiflora edulis Extract in Cultured 3T3L1

### Cell Culture

Mouse 3T3-L1 mouse embryo fibroblasts (American Type Culture Collection (ATCC, Rockville, MD, USA) were cultured in DMEM containing 25 mM glucose with 10% heat-inactivated fetal calf serum with antibiotics at 37° C. and 5% CO2. When the cells were 70-80% confluent, they were trypsinized, washed and seeded in 6 well plates at a density of 2×10⁶ cells per well. Cells were induced to differentiate 2 d after reaching confluence (day 0), by supplementing DMEM media containing 10% Fetal Bovine Serum (FBS) along with 1 μg/mL insulin, 0.25 μM dexamethasone, 0.5 mM 1-methyl-3-isobutyl-xanthine (IBMX) and different concentrations of Cyperus rotundus extract and Passiflora edulis extract comprising Piceatannol, Scirpusin B, Scirpusin A Cyperusphenol A and Cyperusphenol B, standardized to contain 6-10% w/w total stilbenes (Composition 1—with tradename CIRPUSINS®) or Cyperus extract standardized to contain 3-5% w/w total stilbenes (Composition 2-CRE) at different concentrations. The composition comprising Piceatannol, Scirpusin B, Scirpusin A Cyperusphenol A and Cyperusphenol B, standardized to contain 6-10% w/w total stilbenes is trademarked as CIRCPUSINS®. From day 3 until day 7, cells were maintained in progression media supplemented with 1 μg/mL insulin and different concentrations of the composition comprising CIRPUSINS®, compared to Composition 2-CRE. Untreated cells and undifferentiated cells grown in FCS media were taken as A positive and negative controls for the experiment. To study inhibition of adipogenesis, cells were plated in 48 well plate at a density of 10000 cells per well and differentiated as mentioned earlier. Quantification for amount of triglycerides accumulated in adipocytes was done by Oil red O staining Cell viability was measured using sulforhodamine-B (SRB) assay (Orellana & Kasinski, 2016, Assay in Cell Culture to Investigate Cell Proliferation. Bio Protoc. 2016 Nov. 5; 6(21):e1984. doi: 10.21769/BioProtoc.1984.).

RNA Extraction and qRT-PCR

RNA was extracted from 3T3-L1 cells using the trizol method and treated with DNAse to remove any traces of DNA (Au-Tan, Au-Pepin, & Au-Lavoie, 2018). First-strand cDNA was prepared from RNA samples using oligo dT primers and Superscript III reverse transcriptase (cDNA synthesis kit, Invitrogen™). Quantitative real-time PCR (qRT-PCR) was performed with SYBR Green I fluorescent dye using Light cycler 96′1 according to the manufacturer's instructions (Light Cycler® FastStart DNA Master SYBR Green I, Roche). The α-actin gene was used as the housekeeping gene. The following primers were used:

UCP1
(SEQ ID: 1)
F-AGGCTTCCAGTACCATTAGGT
(SEQ ID: 2)
R-CTGAGTGAGGCAAAGCTGATTT
PGC1α
(SEQ ID: 3)
F-TATGGAGTGACATAGAGTGTGCT
(SEQ ID: 4)
R-CCACTTCAATCCACCCAGAAAG
PRDM16
(SEQ ID: 5)
F-CAGCACGGTGAAGCCATTC
R-GCGTGCATCCGCTTGTG
BMP7
(SEQ ID: 7)
F-CCAAAGAACCAAGAGGCCC
R-GCTGCTGTTTTCTGCCACACT
β-Actin
(SEQ ID: 9)
F-GAAGTCCCTCACCCTCCCAA
(SEQ ID: 10)
R-GGCATGGACGCGACCA

### Gene Expression for Adipocyte Browning

The mRNA levels of transcripts associated with energy metabolism (PGC-la), factors causing adipocyte browning (PRDM16), and secretary protein inducing BAT differentiation (BMP7), were assessed in the compositions treated cells compared to control. Fold change in expression was calculated in comparison to untreated differentiated cells, using the DDCT method

### Results

### Gene Expression

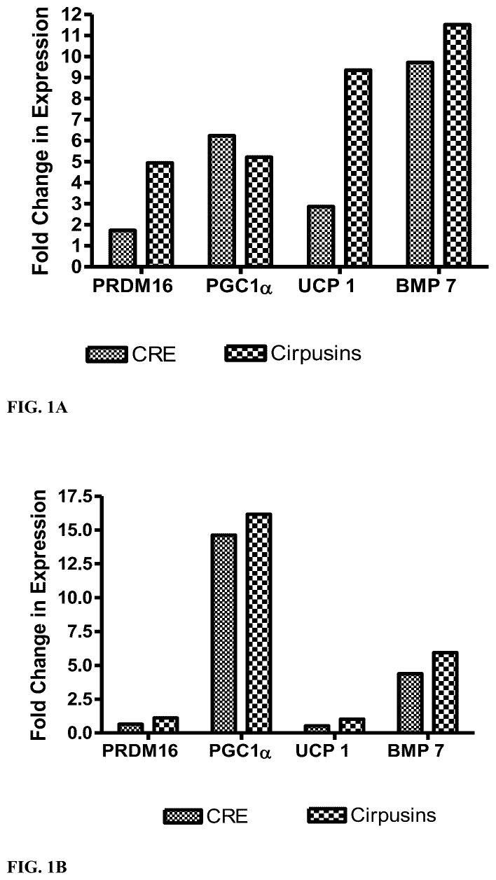

The fold change in the expression of genes related to BAT, compared between CRE (Cyperus extract) and Cirpusins (Composition of Instant invention) are presented in FIG. 1A (at 3.125 μg/ml) and 1B (at 6.25 μg/ml). The results indicated that the fold change in the expression of genes in cells treated with CIRPUSINS was better compared to CRE (Cyperus rotundus extract standardized to contain 3-5% total stilbenes) (TABLE 1), indicating that the composition comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes, performs better in increased energy expenditure compared to Cyperus rotundus extract standardized to contain 3-5% total stilbenes. Thus, further studies of adipogenesis inhibition and preventing weigh gain in mammals were carried out for the composition comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes.

TABLE 1
Fold Change in expression compared to
differentiated and untreated cells

| | | Fold Change in expression compared to | | | |
|---|---|---|---|---|---|
| | | differentiated and untreated cells | | | |
| Sample Concentration | | | PGC1 | | |
| (μg/ml) | | PRDM16 | ALPHA | UCP 1 | BMP 7 |
| CRE | 3.125 | 0.65 | 14.62 | 0.53 | 4.38 |
| | 6.25 | 1.73 | 6.23 | 2.87 | 9.71 |
| CIRPUSINS | 3.125 | 1.11 | 16.17 | 1.03 | 5.94 |
| | 6.25 | 4.94 | 5.21 | 9.35 | 11.51 |

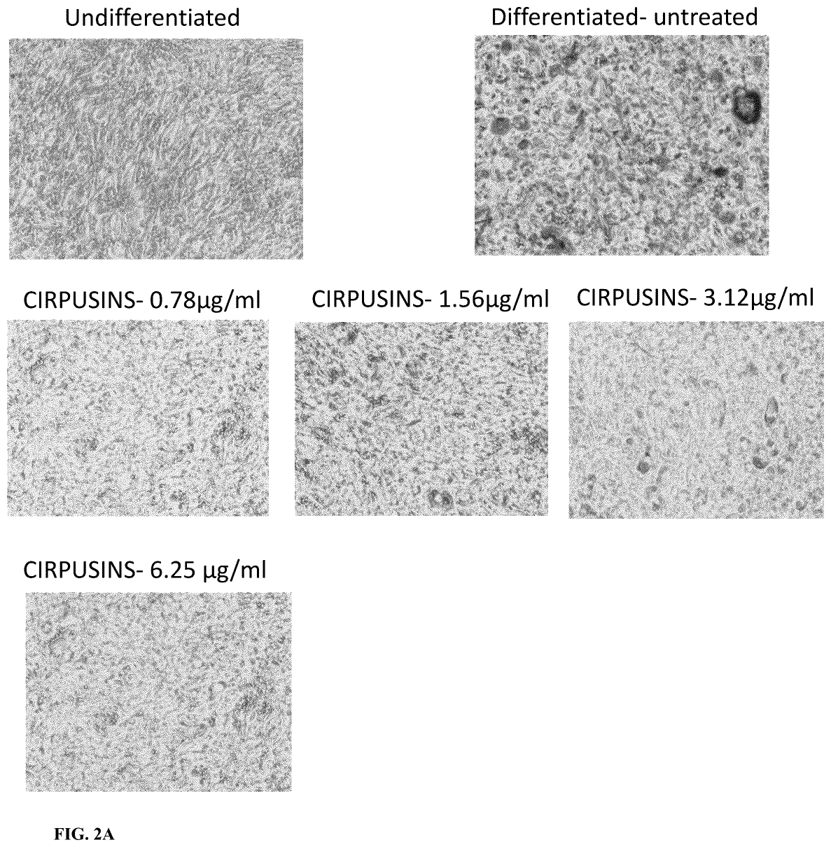

The composition comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes showed a dose dependant inhibition of adipogenesis (FIGS. 2A and 2B)

### Example 3: Anti-Obesity Studies

### Methods

| | |
|---|---|
| Test system: Animal Species | Mice |
| Strain | C57 |
| Sex | Male |
| Justification for selection of | C57 Mice are recommended species |
| species | for conducting pharmacological study |
| | especially anti-obesity among the |
| | rodents and hence Anti-obesity study |
| | has been carried out in C57 Mice models. |
| No. of animals/group | 8 animals/Group. |
| Body weight range at receipt | 15-20 g |
| Age at treatment | 6-8 weeks |

Animals were housed under standard laboratory conditions, air-conditioned with adequate fresh air supply (12-15 Air changes per hour), room temperature 20.2-23.5° C. and relative humidity 58-64% with 12 hours fluorescent light and 12 hours dark cycle. The temperature and relative humidity was recorded once daily. Maximum of four animals were housed in a standard polypropylene cage with stainless steel mesh top grill having facilities for holding food and drinking water in water bottled fitted with stainless steel sipper tube. Clean sterilized paddy husk was provided as bedding material.

The animals were fed with Normal diet (Group-I) and High fat diet (Group-II to V) throughout the acclimatization and experimental period. Water was provided along with High Fat Diet to the animals throughout the acclimatization and experimental period. Water from water filter cum purifier was provided in animal feeding bottle with stainless steel sipper tubes.

### Study Groups

TABLE 2
Groups for anti-obesity studies

| Groups | Treatment |
|---|---|
| G1 - Control | Mice were fed with Normal diet for 120 Days. |
| G2 - HFD | Mice were fed with high fat diet for 120 Days. |
| G3 - HFD + | Mice were fed with high fat diet for 16 weeks |
| CIRPUSINS | were administered with Cyprus extract |
| (50 mg/kg | (50 mg/kgbw) for 120 Days. |
| body weight) | |
| G4 - HFD + | Mice were fed with high fat diet for 16 weeks |
| CIRPUSINS | were administered with Cyprus extract |
| (100 mg/kg | (100 mg/kgbw) for 120 Days. |
| body weight) | |
| GS - HFD + | Mice were fed with high fat diet for 16 weeks |
| CIRPUSINS | and the mice were administered with Cyprus |
| (200 mg/kg | extract (200 mg/kgbw) for 120 Days. |
| body weight) | |

Body weight of the animals was recorded in all the days of experimental period. At the end of the experimental period, the animals were sacrificed by cervical dislocation. Blood was collected, Serum was separated by centrifugation and used for the analysis of biochemical parameters. The organs such as Liver and Fat Pads (Retroperitoneal, Peri-gonadal and Mesenteric) were dissected out and washed in phosphate buffered saline.

The following parameters were studied:
1. Measurement of Body weight
2. Estimation of Blood Glucose (GOD/POD Method)
3. Estimation of Uric acid (URICASE/POD Method)
4. Estimation of AST (Enzyme Kinetic Method)
5. Estimation of ALT (Enzyme Kinetic Method)
6. Estimation of Cholesterol (CHOD/POD Method)
7. Estimation of Triglycerides (GPO/POD Method)
8. Estimation of HDL Cholesterol (HDL Cholesterol Direct Reagent Kit, Beacon)
9. Determination of LDL Cholesterol (LDL Cholesterol Direct Reagent Kit, Beacon)

### Results

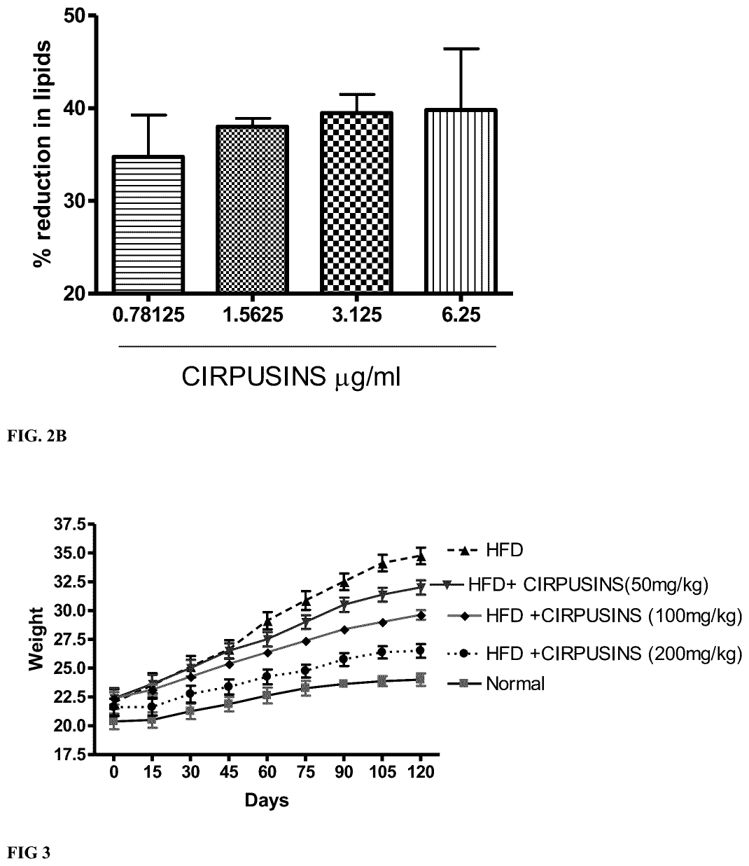

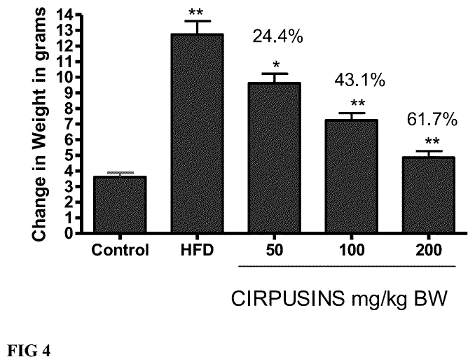

The composition showed a dose dependant decrease in the body weight of the mice (FIG. 3 and FIG. 4). The weight gain induced by high fat diet was significantly reduced by the composition. At the highest dose of 200 mg/kg bw, the reduction was 61.7%

Significant reduction in retroperitoneal, perigonadal and mesenteric fat was also observed in the animals treated with the composition.

TABLE 3
Visceral fat weights in mice treated with CIRPUSINS

| | Retroperitoneal | Perigonadal | Mesenteric |
|---|---|---|---|
| | Fat (g | Fat (g | Fat (g |
| | wet tissue) | wet tissue) | wet tissue) |
| G1 - Control | 0.308 ± 0.01 | 0.413 ± 0.01 | 0.350 ± 0.02 |
| G2 - HFD | 0.848 ± 0.05 | 1.208 ± 0.09 | 0.837 ± 0.01 |
| G3 - HFD + CIRPUSINS | 0.817 ± 0.04 | 1.073 ± 0.03 | 0.784 ± 0.24 |
| (50 mg/kg body weight) | | | |
| G4 - HFD + CIRPUSINS | 0.606 ± 0.06 | 0.869 ± 0.03 | 0.595 ± 0.03 |
| (100 mg/kg body weight) | | | |
| G5 - HFD + CIRPUSINS | 0.417 ± 0.03 | 0.557 ± 0.01 | 0.388 ± 0.01 |
| (200 mg/kg body weight) | | | |

Among the biochemical parameters, the CIRPUSINS composition improved the lipid profile by decreasing the elevated levels of total cholesterol, LDL and triglycerides and increasing HDL cholesterol (Table 4)

TABLE 4
Lipid profile

| | | HDL | LDL | TG |
|---|---|---|---|---|
| Groups | Cholesterol | (mg/dl) | (mg/dl) | (mg/dl) |
| G1 - Control | 83.88 ± 2.75 | 64.38 ± 2.92 | 6.73 ± 0.37 | 105.63 ± 2.00 |
| G2 - HFD | 190.63 ± 2.62 | 24.38 ± 2.77 | 76.45 ± 2.69 | 234.38 ± 2.67 |
| G3 - HFD + | 182.13 ± 2.75 | 26.63 ± 1.69 | 69.69 ± 3.34 | 178.25 ± 2.49 |
| CIRPUSINS | | | | |
| (50 mg/kg | | | | |
| body weight) | | | | |
| G4 - HFD + | 169.63 ± 2.97 | 35.25 ± 2.49 | 43.48 ± 2.02 | 159.63 ± 2.67 |
| CIRPUSINS | | | | |
| (100 mg/kg | | | | |
| body weight) | | | | |
| G5 - HFD + | 94.75 ± 2.82 | 55.50 ± 2.56 | 20.08 ± 0.67 | 126.38 ± 2.62 |
| CIRPUSINS | | | | |
| (200 mg/kg | | | | |
| body weight) | | | | |

The composition also improved the elevated levels of glucose, Uric acid, and the liver enzymes (table 5)

TABLE 5
Biochemical parameters

| | Glucose | Uric Acid | | |
|---|---|---|---|---|
| Groups | (mg/dl) | (mg/dl) | AST(U/L) | ALT(U/L) |
| G1 - Control | 89.50 ± 3.82 | 4.06 ± 0.16 | 30.50 ± 2.45 | 25.50 ± 1.51 |
| G2 - HFD | 153.13 ± 3.00 | 5.91 ± 0.24 | 53.38 ± 3.81 | 54.25 ± 1.67 |
| G3 - HFD + | 148.50 ± 2.45 | 5.49 ± 0.31 | 43.25 ± 2.87 | 48.75 ± 1.04 |
| CIRPUSINS | | | | |
| (50 mg/kg | | | | |
| body weight) | | | | |
| G4 - HFD + | 123.00 ± 3.82 | 5.34 ± 0.27 | 38.38 ± 2.67 | 41.25 ± 1.04 |
| CIRPUSINS | | | | |
| (100 mg/kg | | | | |
| body weight) | | | | |
| G5 - HFD + | 92.38 ± 3.11 | 4.63 ± 0.27 | 31.38 ± 1.06 | 34.75 ± 1.67 |
| CIRPUSINS | | | | |
| (200 mg/kg | | | | |
| body weight) | | | | |

Overall, The results suggest that comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes (CIRPUSINS®) is efficacious in controlling obesity in animals.

### Example 3: Formulations Containing CIRPUSINS®

The composition is formulated along with pharmaceutically/nutraceutically acceptable excipients, adjuvants, diluents, stabilizing agents, dispersible gums, bioavailability enhancers or carriers and administered orally in the form of tablets, capsules, syrups, gummies, powders, suspensions, emulsions, chewables, candies or eatables.

In a related aspect the bioavailability enhancer is selected from the group of piperine (BioPerine®), quercetin, garlic extract, ginger extract, and naringin. In another related aspect, the stabilizing agent is selected from the group consisting rosmarinic acid, butylated hydroxyanisole, butylated hydroxytoluene, sodium metabisulfite, propyl gallate, cysteine, ascorbic acid and tocopherols. In yet another related aspect, the dispersible gums are selected from the group consisting of Agar, Alginate, Carrageenan, Gum Arabic, Guar Gum, Locust Bean Gum, Konjac Gum, Xanthan Gum and Pectin.

Tables 6-10 provide illustrative examples of nutraceutical formulations containing CIRPUSINS

TABLE 6
Powder Formulation
Active Ingredients
CIRPUSINS ®
Excipients
Microcrystalline cellulose, Dextrin

TABLE 7
Tablet
Active Ingredients
CIRPUSINS ®
Excipients
Microcrystalline cellulose, Colloidal silicon dioxide, Magnesium stearate,
BioPerine ®, Polyvinylpyrrolidone/starch/Hydroxy propyl methyl cellulose,
Hydroxy propyl cellulose

TABLE 8
Capsule
Active Ingredients
CIRPUSINS ®
Excipients
Microcrystalline cellulose, BioPerine ®

TABLE 9
Powder
Active Ingredients
CIRPUSINS ®
Excipients
BioPerine ®, Microcrystalline cellulose, Dextrin

TABLE 6
Gummy formulation
Active Ingredients
CIRPUSINS ®
Excipients
BioPerine ®, Gelatin (270 Bloom Mesh 10), Refined Sugar, Glucose
Corn Syrup, Citric Acid, Lactic Acid, Water, Natural Mango
Flavor M38630, Tartaric Acid, Refined Sugar

TABLE 7
Candy formulation
Active Ingredients
AC3 complex (Curcumin BD3 Complex)
Excipients
BioPerine ®, Sucrose, Liquid Glucose, Flavoring agent, Menthol, Acidulants
(Citric acid/Tartaric Acid/Maleic Acid), Purified water

The above formulations are merely illustrative examples, any formulation containing the above active ingredient intended for the said purpose will be considered equivalent.

Other modifications and variations of the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention and is to be interpreted only in conjunction with the appended claims.

SEQUENCE LISTING
SEQ ID NO 1: FORWARD PRIMER FOR UCP1
AGGCTTCCAGTACCATTAGGT
SEQ ID NO 2: REVERSE PRIMER FOR UCP1
CTGAGTGAGGCAAAGCTGATTT
SEQ ID NO 3: FORWARD PRIMER FOR PGC1 ALPHA
TATGGAGTGACATAGAGTGTGCT
SEQ ID NO 4: REVERSE PRIMER FOR PGC1 ALPHA
CCACTTCAATCCACCCAGAAAG
SEQ ID NO 5: FORWARD PRIMER FOR PRDM16
CAGCACGGTGAAGCCATTC
SEQ ID NO 6: REVERSE PRIMER FOR PRDM16
GCGTGCATCCGCTTGTG
SEQ ID NO 7: FORWARD PRIMER FOR BMP7
CCAAAGAACCAAGAGGCCC
SEQ ID NO 8: REVERSE PRIMER FOR BMP7
GCTGCTGTTTTCTGCCACACT
SEQ ID NO 9: FORWARD PRIMER FOR BETA ACTIN
GAAGTCCCTCACCCTCCCAA
SEQ ID NO 10: REVERSE PRIMER FOR BETA ACTIN
GGCATGGACGCGACCA

We claim:

1. A composition comprising Cyperus rotundus extract and Passiflora edulis extract comprising Piceatannol, Scirpusin B, Scirpusin A, Cyperusphenol A and Cyperusphenol B, standardized to contain 6-10% w/w total stilbenes.

2. The composition as in claim 1, wherein the Cyperus rotundus extract is standardized to contain 0.1-5% total stilbenes and Passiflora edulis extract is standardized to contain 15-25% total stilbenes.

3. The composition as in claim 1, wherein the Cyperus rotundus extract and Passiflora edulis extract are in the ratios ranging from 1:5 to 5:1.

4. The composition as in claim 1, wherein the Cyperusphenol A include both the meso and dl isomers.

5. The composition as in claim 1, wherein the composition comprising Cyperus rotundus extract and Passiflora edulis extract is obtained by the process comprising steps of:
a) Powdering tubers of Cyperus rotundus into a fine mesh;
b) Refluxing with 4 volumes of methanol and heating at 65-70° C. for 3-4 hours and filtering the solution to obtain a clear extract;
c) Repeating the process of step b) twice and combining the three extracts;
d) Concentrating the extract of step c) to obtain as extract containing 0.1-5% total stilbenes;
e) Powdering seeds of Passiflora edulis into a fine mesh:
f) Refluxing with 4 volumes of hexane and heating at 65-70° C. for 3-4 hours and filtering the solution;
g) Repeating the process of step f) twice and combining the three hexane extracts;
h) Concentrating the combined extract of step g) by distilling the solvent to obtain a clear Marc;
i) Adding 4 volumes of ethanol to the Marc of step h) and heating at 70-75° C. and filtering;
j) Repeating the process of step i) twice and combining the three ethanol extracts to obtain a clear ethanol extract of the Passiflora edulis seeds;
k) Dissolving the ethanol extract of step j) in 6 volumes of water;
l) Charging with 6 volumes of ethyl acetate and stirring for 5 minutes and allowing to settle for 1 hour;
m) Collecting the ethyl acetate layer and washing with water;
n) Repeating the process of steps 1) and m) two more times and combining all ethyl acetate layers;
o) Concentrating the ethyl acetate layer by heating at 65-75° C. to obtain Passiflora edulis seed extract containing 15-25% total stilbenes;
p) Mixing the Cyperus rotundus extract and Passiflora edulis seed extract in the ratios of 1:5, 2:4, 3:3, 4:2, 2:1 and 5:1 respectively to obtain a composition comprising Cyperus rotundus extract and Passiflora edulis extract comprising Piceatannol, Scirpusin B, Scirpusin A Cyperusphenol A and Cyperusphenol B, standardized to contain 6-10% w/w total stilbenes.

6. The composition as in claim 1, wherein the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.
